(19) United States
(12) Patent Application Publication
(10) Pub. No.: US 2008/0254010 A1
(43) Pub. Date: Oct. 16, 2008

(54) Controlling Clostridium difficile-Associated Disease in the Gastrointestinal Tract

(76) Inventors: Joseph Myron Sasser, Newark, DE (US); Katrina Shoemaker Cousin, Bear, DE (US)

Correspondence Address: FISH & RICHARDSON P.C., P.O. BOX 1022, MINNEAPOLIS, MN, 55440-1022, US

(21) Appl. No.: 12/047,126

(22) Filed: Mar. 12, 2008

Related U.S. Application Data
(60) Provisional application No. 60/906,470, filed on Mar. 12, 2007.

(51) Int. Cl. A61K 35/74; A61K 31/195; A61K 31/7016; A61K 33/00; A61K 31/718; A61K 31/66; A61K 31/20; A61P 31/04; A61K 31/215; A61K 31/56; A61K 31/7076; A61K 31/7042; A61K 31/4164

(52) U.S. Cl. 424/93.44; 514/561; 514/53; 424/600; 514/60; 514/182; 514/46; 514/31; 514/398; 424/93.45; 424/93.51; 514/114; 514/560; 514/546

(57) ABSTRACT

This document provide materials and methods for treating patients infected with a spore-forming bacteria and materials and methods for preventing development of Clostridium difficile-associated disease.

### CROSS-REFERENCE TO RELATED APPLICATIONS

This application claims the benefit of U.S. Provisional Application Ser. No. 60/906,470, filed Mar. 12, 2007, the contents of which is incorporated herein.

### TECHNICAL FIELD

The present application relates to methods and materials for controlling Clostridium difficile disease in the gastrointestinal tract. More specifically, the present application relates to methods for controlling Clostridum difficile by inducing spores of Clostridium difficile to germinate during antibiotic therapy.

### BACKGROUND

Clostridium difficile (C. difficile) is a Gram-positive spore-forming bacterium that produces exotoxins that are pathogenic to humans. C. difficile-associated disease (CDAD) usually includes diarrhea, fever, cramps, fecal leukocytes and may include hypoalbuminemia and, in advanced cases, pseudomembranous colitis. See, Bartlett et al. New Engl. J. Med. 1978, 298L531-534. The tissue culture assay is considered the most sensitive assay for detecting C. difficile infection. Due to ease of use and speed of detection, immunoassays detecting toxin A or toxins A and B also are commonly used.

Risk factors associated with CDAD include antibiotic treatment, hospitalization, and advanced age. See Bartlett et al., Ann. Intern. Med. 2006, 145:758-764. Numerous antibiotics have been implicated in causation of CDAD, including clindamycin, ampicillin, cephalosporins and fluoroquinolones. Antibiotic use can disrupt the protective microflora present in the intestinal tract, which typically prevent colonization and subsequent infection by opportunistic pathogens such as C. difficile. After exposure to broad spectrum antibiotics, it can take the normal microflora up to six weeks to recover. See McFarland, J. Medic. Microbiol. 2005, 54:101-111.

Another major contributing factor to CDAD is the widespread contamination of hospitals, leading to 20%-40% colonization in hospitalized adults vs. 2%-3% in healthy adults. See Kim et al., J. Infect. Dis 1981, 143:42-50. CDAD is the leading cause of nosocomial (i.e., hospital acquired) gastrointestinal illness. See McFarland, 2005, supra. By one estimate, a patient in the hospital for 1-2 weeks has a 13% chance of contracting CDAD; if hospitalized for more than 4 weeks, the rate increases to 50%. Johnson and Gerding, Clin. Infect. Dis. 1998, 26:1027-1036. Mild cases of CDAD may resolve without treatment other than withdrawing antibiotic treatment or switching to an antibiotic with a narrower spectrum of activity (e.g., betalactams, macrolides or metronidazole). With persistent symptoms, metronidazole or vancomycin typically is used for therapy. A need exists for methods and compositions for treating and/or preventing CDAD.

### SUMMARY

Disclosed are materials and methods for treating patients infected with spore-forming bacteria and materials and methods for preventing development of CDAD. The methods include administering an antibiotic and a spore germinant in amounts and for durations effective for treating the patient. Materials and methods also are disclosed for inhibiting toxin production and for adsorbing or absorbing toxin(s) and causing elimination with fecal matter.

In one aspect, the present application relates to a method for treating a patient infected with spore-forming bacteria (C. difficile or Clostridium perfringens). The method includes administering to the patient an antibiotic and a spore germinant in amounts and for durations effective for treating the patient. The spore germinant can be an amino acid such as L-alanine, L-asparagine, L-cysteine, and L-glutamine, or a derivative thereof such as N-(L-α-aspartyl)-L-phenylalanine. The spore germinant can be L-lactate or lactose, carbonic acid, a compound that adsorbs lipids such as a starch or charcoal, a bile salt such as taurocholate, inosine, or a mixture of L-alanine, L-lactose, and sodium bicarbonate. The antibiotic can be vancomycin or metronidazole. The antibiotic and the spore germinant can be administered simultaneously. The antibiotic can be administered first and the spore germinant can be administered second. The spore germinant can be administered first and the antibiotic can be administered second. The spore germinant can be administered in increasing amounts over a duration of four days.

A method of the present application further can include administering to the patient a) an amount of an agent effective for inhibiting Clostridium difficile toxin production or b) an amount of an agent effective for binding and eliminating Clostridium difficile toxin from the patient. An agent effective for inhibiting Clostridium difficile toxin production can be calcium acetate, calcium gluconate, or calcium saccharate. An agent effective for binding and eliminating Clostridium difficile toxin can be Tolevamer or Cholestyramine.

A method of the present application further can include administering a probiotic organism to the patient. The probiotic organism can be Lactobacillus, Lactococcus, Pediococcus, or Saccharomyces boulardii.

A method of the present application further can include administering to the patient an agent effective for inhibiting spore germination, wherein the agent is administered after discontinuing administration of the spore germinant. An agent effective for inhibiting spore germination can be lecithin, linolenic acid, sorbate, or 1-kestose.

In another aspect, the present application features a method of preventing CDAD in a patient receiving an antibiotic. The method can include administering a spore germinant to the patient in an amount and for a duration effective to prevent CDAD. The spore germinant can be L-alanine. A method of the present application further can include controlling the patient's diet and/or monitoring the patient for symptoms of CDAD.

The present application also features a method of preventing CDAD in a patient receiving an antibiotic. The method includes administering an inhibitor of spore germination to the patient in an amount and for a duration effective to prevent CDAD.

In another aspect, the present application features a composition that includes an antibiotic and a spore germinant. The spore germinant can be L-alanine. The antibiotic can be vancomycin or metronidazole. The composition further can include an agent effective for inhibiting Clostridium difficile toxin production or an agent effective for binding and eliminating Clostridium difficile toxin from said patient.

In yet another aspect, the present application features an article of manufacture that includes an antibiotic, a spore germinant, and a label or package insert indicating that the antibiotic and spore germinant are useful for preventing CDAD in patient at risk for developing CDAD. The article of manufacture further can include an agent effective for inhibiting Clostridium difficile toxin production or an agent effective for binding and eliminating Clostridium difficile toxin from the patient. The article of manufacture further can include an agent effective for inhibiting spore germination.

The present application also features a method for treating a patient infected with spore-forming bacteria. The method includes administering to the patient an antibiotic and L-alanine in amounts and for durations effective for treating the patient.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

In general, materials and methods are disclosed for treating patients infected with spore-forming bacteria such as C. difficile or Clostridium perfringens. The methods include administering one or more antibiotics and one or more spore germinants in amounts and for durations effective for treating the patient. The term “spore germinant” as used herein refers to a compound that induces spore germination. Thus, the methods described herein induce spores to germinate, making the bacteria more susceptible to an antibiotic. In particular, for C. difficile, spore germinants induce germination of the spores, which are resistant to virtually all antibiotics used in hospitals, to the vegetative stage, which is susceptible to most broad-spectrum antibiotics and antibiotics effective against Gram (+) bacteria.

### Methods of Treatment or Prevention

Typically, spore germinants and antibiotics are administered to a mammal such as a human patient that is infected with a spore-forming bacteria. Spore germinants also can be administered prophylactically in patients at risk for developing CDAD (e.g., a patient receiving antibiotic treatment in a hospital) to reduce or eliminate infection with C. difficile, prevent development of symptoms of the disease from occurring, delaying onset of disease symptoms, or lessening the severity of subsequently developed disease symptoms. Treatment of a patient infected with spore-forming bacteria can include reducing symptoms of the disease (e.g., reducing diarrhea) or eliminating symptoms of the disease. In either case, spore germinants and antibiotics are administered to the patient in amounts and for durations effective for treating the patient or preventing CDAD.

As used herein, “an amount effective” refers to an amount of a spore germinant and/or antibiotic that reduces the deleterious effects of the spore-forming bacteria without inducing significant toxicity to the host. Effective amounts of spore germinants and antibiotics can be determined by a physician, taking into account various factors that can modify the action of drugs such as overall health status, body weight, sex, diet, time and route of administration, other medications, and any other relevant clinical factors. In some embodiments, an effective amount of spore germinant can range from 0.5 g to 10 g per day(e.g., 0.9 g to 9 g, 1 g to 8 g, 1.5 g to 8.5 g, 2 g to 8 g, 3 g to 7 g, 1 g, 2 g, 3 g, 4 g, 5 g, 6 g, 7 g, 8 g, 9 g, or 10 g per day).

Suitable spore germinants, including combinations of spore germinants, can be identified by comparing germination of spores in the presence and absence of the spore germinants via phase-contrast microscopy. See, Example 1. Non-limiting examples of spore germinants include L-lactate; lactose (as found in dairy products), bicarbonate or carbonate compounds such as sodium bicarbonate; carbon dioxide (e.g., carbonic acid: CO₂ dissolved in water, as is common in “sodas” or “soft drinks” such as cola or some fruit flavored beverages); compounds that adsorb lipid (e.g., starch, such as found in wheat, rice or other grains and potatoes and some other vegetables); charcoal or similar materials of high surface area that may adsorb or absorb fatty acid and lipid materials that may inhibit spore germination; monosaccharides such as fructose, glucose, mannose, or galactose; L-alanine, L-asparagine, L-cysteine, L-glutamine, L-norvatine, L-serine, L-threonine, L-valine, L-glycine, or other amino acid, and derivatives thereof such as N-(L- a-aspartyl)-L-phenylalanine (commonly sold under the trade name of “Aspartame”); inosine; bile salts such as taurocholate; and combinations of such spore germinants. For example, useful spore germinants can include L-alanine alone or in combination with L-lactate; a combination of L-asparagine, glucose, fructose, and potassium ion (AGFK); amino acids such as L-aspargine, L-cysteine, or L-serine alone or in combination with L-lactate; and caramels created by autoclaving monosaccharides or such caramels in combination with amino acids. L-alanine is a particularly useful spore germinant and is considered to be generally recognized as safe (GRAS) by the U.S. Food and Drug Administration.

Any antibiotic that has broad spectrum or Gram positive activity can be used in the methods described herein. For example, cephalosporins; carbapenems such as Imipenem, Meropenem, Ertapenem, Faropenem, Doripenem, and Panipenem/betamipron; glycopeptides such as vancomycin, teicoplanin, telavancin, ramoplanin, and decaplanin; macrolides such as azithromycin (Zithromax, Zitromax), Clarithromycin (Biaxin), Dirithromycin (Dynabac), Erythromycin, and Roxithromycin (Rulid, Surlid,Roxid); penicillins; quinolones; nitroimidazoles (e.g., metronidazole) and tetracyclines can be used in the methods. Non-limiting examples of cephalosporins include Cefacetrile, Cefadroxil; Cefalexin (Keflex®), Cephaloglycin, Cefalonium, Cefaloridine, Cefapirin (Cefadryl®), Cefatrizine, Cefazaflur, Cefazedone, Cefazolin (Ancef®, Kefzol®), Cefradine (Velosef®), Cefroxadine, Ceftezole, Cefaclor (Ceclor®, Distaclor®, Keflor®, Raniclor®), Cefonicid (Monocid®), Cefprozil (cefproxil; Cefzil®, Procef®), Cefuroxime (Ceftin®), Cefuzonam, Cefmetazole, Cefotetan, Cefoxitin, Cefcapene, Cefdaloxime, Cefdinir (Omnicef®), Cefditoren, Cefetamet, Cefixime (Suprax®), Celmenoxime, Cefodizime, Cefoperazone (Cefobid®), Cefotaxime (Claforan®), Cefpimizole, Cefpodoxime (Vantin®), Cefteram, Ceftibuten (Cedax®), Ceftiofur, Ceftiolene, Ceftizoxime (Cefizox®), Ceftriaxone (Rocephin®), Cefclidine, Cefepime (Maxipime®), Cefluprenam, Cefoselis, Cefozopran, Cefpirome, and Cefquinome. Non-limiting examples of penicillins include Amoxicillin (Amoxil®, Prevpac®, Trimox®, Ampicillin (Principen®), Azlocillin, Azlocillin, Cloxacillin, Dicloxacillin, Flucloxacillin, Mezlocillin, Nafcillin, Penicillin, Piperacillin, and Ticarcillin. Non-limiting examples of quinolones include ciprofloxacin (Ciprobay®, Cipro®, Ciproxin®), nalidixic acid (NegGam®, Wintomylon®), oxolinic acid, piromidic acid, pipemidic acid (Dolcol®), enoxacin (Enroxil®, Penetrex®), nadifloxacin, norfloxacin (Noroxin®), ofloxacin (Floxin®, Oxuflox®), pefloxacin, rufloxacin (Uroflox®R), balofloxacin, levofloxacin (Levaquin®), tosufloxacin, clinafloxacin, gemifloxacin (Factive®), moxifloxacin (Avelox®)), and sitafloxacin. For example, nitroimidazoles such as metronidazole, glycopeptides such as vancomycin, clindamycin, ampicillin, cephalosporins, and fluoroquinolones are particularly useful antibiotics.

Antibiotics and spore germinants can be administered by any route, including, without limitation, oral or parenteral routes of administration such as intravenous, intramuscular, intraperitoneal, subcutaneous, intrathecal, intraarterial, nasal, transdermal (e.g., as a patch), or pulmonary absorption. In some embodiments, an antibiotic can be administered intracolonically. An antibiotic or spore germninant can be formulated as, for example, a solution, suspension, or emulsion with pharmaceutically acceptable carriers or excipients suitable for the particular route of administration, including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives, flavorings, sugars, and other additives such as antimicrobials, antioxidants, chelating agents, inert gases, and the like also may be present.

For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration also can be formulated to give controlled release of the compound. In some embodiments, a spore germinant can be administered in three or four doses via a controlled release formulation to provide continuous release primarily in the large intestine. For example, three or four doses of a controlled release formulation can be used to deliver 0.9 g to 9 g of L-alanine per day to a patient over a duration of three to four days.

Nasal preparations can be presented in a liquid form or as a dry product. Nebulised aqueous suspensions or solutions can include carriers or excipients to adjust pH and/or tonicity.

In some embodiments, an antibiotic and a spore germinant are administered simultaneously (e.g., in same or different formulations). In some embodiments, a spore germinant is administered first and an antibiotic is administered second. In some embodiments, an antibiotic is administered first and a spore germinant is administered second. For example, spore germinants can be administered during antibiotic therapy for C. difficile infection, including an initial or recurrent C. difficile infection. In some embodiments, vancomycin and metronidazole are antibiotics that can be used to treat C difficile infection. In another example, spore germinants can be administered during routine antibiotic therapy in a hospital to prevent development of CDAD. For example, an oral antibiotic such as a cephalosporin can be administered to prevent infection from a surgery and a spore germinant can be subsequently administered.

As individual C. difficile spores may respond differently to different concentrations of the spore germinant, the spore germinant can be administered in increasing amounts over a duration of days (e.g., four or five days) over the course of antibiotic therapy. For example, an antibiotic can be administered to a patient for a period of time (e.g., one to three days) sufficient to kill much of the population of C. difficile vegetative cells and competing microflora. Following this initial period of antibiotic administration, spore germinants can be administered in increasing amounts for a period of time (e.g., three to six days) sufficient to induce spore germination. In one embodiment, L-alanine or a mixture of spore germinants (e.g., L-alanine, L-lactate, and sodium bicarbonate) that induce a modest percentage of spore germination can be used. Following this initial dose, increasing amounts of spore germinants can be administered each day for the next two to five days. This may avoid a rapid increase in toxin production resulting from the germination of the spores to the antibiotic susceptible vegetative phase.

After inducing spore germination, further germination of any residual spores of the organism can be inhibited by administering one or more agents effective for inhibiting spore germination, allowing other common GI tract bacteria (e.g. Bacteroides, Bzfidobacterium, etc.) to heavily colonize the GI tract and to competitively inhibit the remaining spores (e.g., C. difficile spores) when-and-if they do germinate into the vegetative state. As such, C. difficile can be suppressed and incidence or recurrence of CDAD can be reduced. Agents that can inhibit spore germination include, but are not limited to D-alanine (an inhibitor of L-alanine induction of spore germination); D-lactate (an inhibitor of L-lactate induction of spore germination); lecithin (e.g., egg or soy lecithin); lipids such as cooking oils, fatty alcohols, aldehydes and the like; sorbic acid and salts of sorbic acid; soluble fiber (e.g., fructo-oligosaccharides (FOS) such as 1-kestose, nystose, fructosylnystose, or bifurcose); and fatty acids. 1-kestose is a particularly useful agent that inhibits spore germination and is considered to be GRAS by the U.S. Food and Drug Administration. Examples of fatty acids include oleic acid, linoleic acid, linolenic acid, docosahexanoic acid, eicosapentacnoic acid, stearic acid, and other fatty acids that can be normal dietary components, can be derived from plant (e.g., vegetable oils), animal or microbial sources, or can be synthesized. Lecithin, linoleic acid, or linolenic acid, and combinations thereof (e.g., lecithin and linoleic acid, lecithin and linolenic acid, linoleic acid and linolenic acid, or lecithin, linoleic acid and linolenic) are particularly useful. Commercial sources of fish oil, flax seed oil, evening primrose oil (or other source of gamma-linolenic acid), and similar dietary supplements also can be used. As starch may counteract the action of fatty acids and lipids, it should be reduced in intake during the process of inhibition of spore germination.

In some embodiments, methods described herein further can include administering an agent effective for inhibiting C. difficile toxin production, and/or an agent effective for binding and eliminating C. difficile toxin from the patient. For example, agents that reduce phosphate levels in the GI tract can be used to inhibit toxin production. Calcium acetate, calcium gluconate, calcium saccharate and similar calcium compounds (but not calcium lactate) are non-limiting examples of agents that can be used to lower phosphate levels in the GI tract. Such calcium compounds are available to treat excess levels of phosphate in the body as they form the relatively insoluble calcium phosphate and such compound would not stimulate toxin production, as do more soluble forms of inorganic phosphate. Such agents can be administered before, during, or after the spore germinant is administered. In some embodiments, it may be particularly useful to administer such agents before or with the spore germinants, such that the agents are available to inhibit or eliminate the toxin(s) at the beginning of vegetative growth and potential toxin release. As the compounds are, by themselves, unlikely to have an adverse effect on the patient in the short term, the dosage should be sufficient to cause a high level of inhibition of toxin production and/or elimination.

Agents that can bind and eliminate toxin include ion exchange materials. For example, Tolevamer, a soluble , high-molecular weight, anionic polymer (>400 kD) that noncovalently binds C. difficile toxins can be used. See, Louie et al., Clin. Infect. Dis. 43:411-20 (2006). Alternatively, cholestyramine (Questran®, Questran Light®, Cholybar®), a bile acid sequestrant can be used to bind and eliminate toxins. In embodiments in which vancomycin is used, cholestyramine should be avoided as it can bind to vancomycin.

In some embodiments, probiotics also can be administered to the patient. As used herein, probiotics refers to mono- or mixed cultures of live microorganisms that can help reestablish normal flora in the GI tract. Probiotics may enhance the immune response, elicit production of enzymes that degrade toxins and/or block attachment sites to the colon. See, McFarland, 2005, supra. Non-limiting examples of probiotic organisms include those in the genera Lactobacillus, Lactococcus, and Pediococcus, Saccharomyces boulardii, and related bacteria and yeast.

In some embodiments, the methods of the present application can include modifying the patient's diet to remove or limit compounds that may directly or indirectly induce spore germination and/or toxin production. For example, the amounts of the following foods can be controlled: dairy products containing L-lactose or L-lactate; foods containing significant levels of starch, e.g., wheat, rice, potatoes, etc.; foods containing significant levels of phosphate such as soft drinks (e.g. colas), chocolate, goods baked using baking powder, and processed meats or cheese; and foods that induce production and/or release of bile salts into the GI tract should be avoided.

Methods described herein also can include monitoring the patient, for example, to identify symptoms of CDAD or to determine if CDAD is improving with treatment. Any method can be used to monitor the patient. For example, time to resolution of diarrhea can be monitored. Alternatively, number of stools, stool consistency, and abdominal discomfort can be monitored. In addition, C. difficile infection can be monitored by a tissue culture assay or by an immunoassay for detecting toxins A and B.

### Articles of Manufacture

Spore germninants can be combined with packaging material and sold as a kit for preventing CDAD in patients at risk or treating patients infected with spore-forming bacteria. Components and methods for producing articles of manufactures are well known. The articles of manufacture may combine one or more spore germinants (e.g., L-alanine alone or in combination with L-lactose and sodium bicarbonate) or antibiotics as described herein. Spore germinants and antibiotics can be in different pharmaceutical compositions or can be in the same pharmaceutical composition.

In addition, the articles of manufacture may further include reagents such as buffers, pharmaceutical carriers, growth media, germination inhibitors, an agent that binds toxin (e.g., Tolevarner), a probiotic, and/or other useful reagents for preventing CDAD or treating patients infected with spore-forming bacteria. For example, an article of manufacture can include L-alanine and an antibiotic such as vancomycin or metronidazole. Such an article of manufacture further can include an agent effective for inhibiting spore germination (e.g., 1-kestose), an agent effective for inhibiting C. difficile toxin production (e.g., calcium acetate, calcium glucanate, or calcium saccharate), and/or an agent effective for binding and eliminating C. difficile toxin (e.g., Tolevamer). The spore germinants or antibiotics can be in a container, such as a plastic, polyethylene, polypropylene, ethylene, or propylene vessel. Instructions describing how the various reagents are effective for preventing CDAD or treating patients infected with spore-forming bacteria also may be included in such kits.

The invention will be further described in the following examples, which do not limit the scope of the invention described in the claims.

### EXAMPLE 1

### Induced Germination of C. difficile

Spores of C. difficile can be induced to germinate into the antibiotic-susceptible vegetative stage.

Inoculum. Clostridium difficile(ATCC 9689) was grown on brucella agar at 35° C. for 7 days to obtain a high percentage of sporulation. Spores were harvested into deionized water, an equal volume of 95% ethanol was added, and the suspension was vortexed vigorously at 10-minute intervals for 1 h to kill the vegetative cells. Debris from the vegetative cells was removed by layering the crude spore preparation onto a 30%-50%-70% step gradient of polyethylene glycol (PEG, 1,000) and centrifuging at 3,000-×g. A sucrose gradient also can be used to purify the spores. More than 99% of the vegetative cellular debris did not penetrate the 70% layer, providing a clean spore preparation. The spores then were washed twice by centrifugation in phosphate buffer to remove the PEG and adjusted to ca. 10⁷ CFU/ml.

Testing of Compounds for Stimulation of Germination or Inhibition of Germination. Ten μl of spore suspension was placed on a sterile cover slip and allowed to air-dry in an open sterile petri dish base. Meanwhile, ca. 150 μl of water-agar (2% agar in DI water, used at 55° C.) containing the test compound (e.g., L-alanine, L-lactate, or L-asparagine) was applied to the surface of a sterile microscope slide in a pattern just smaller than the 22-x-22 mm cover slip. The water-agar was allowed to dry briefly prior to the cover slip being inverted, with the spores being in contact with the agar containing the germinant or inhibitor. Additional water-agar was placed in contact with the edge of the cover slip and the surface of the slide to fill any void space under the cover slip. “Vaspar” (a mixture of petroleum jelly and paraffin) heated to melting point (ca. 55° C.) was “painted” around the edge of the cover slip (using a fine point artist's brush) to make a seal.

Germination was assessed using phase-contrast microscopy by loss of birefringence of the spores. Under phase-contrast microscopy, light spores are not germinated while dark spores are germinated.

In one series of experiments, spore germination was compared between spores incubated with 15 μg/mL L-alanine, spores incubated in the presence of equal concentrations of L-alanine and D-alanine, and untreated control. D-alanine is a specific antagonist of L-alanine. Spore germination was assessed 17 hours after treatment. In spores treated with L-alanine, approximately 79% of the spores germinated. In contrast, only 10% of the untreated spores or spores treated with both L- and D-alanine germinated. The similar percent germination in the control and D-alanine treatments indicates that approximately 10% of the spores were predetermined to germinate without inducement by L-alanine or other treatment.

### EXAMPLE 2

### Inhibition of Germination of Spores and/or Inhibition of Growth

An inoculum of C. difficile spores was prepared and tested as described in Example 1, except that the growth medium of Duncan and Foster (Appl. Microbiol. 16:406-411 (1968)) was substituted for the water-agar to determine effects of compounds on growth. The growth medium contained 20 g proteose peptone, 2.5 g Na₂HP0₄, 1.0 g NaCl, 0.05 g MgS0₄, 3.0 g fructose, 10 g agar, and 500 ml DI water. Lecithin or linolenic acid were added to determine effect on germination and growth. Lecithin in aqueous suspension was added to the growth medium (to make it 0.7 mM) prior to inoculation with spores that had dried on the cover slip. Linolenic acid was dissolved in methanol and added to the medium at 0.7 mM, prior to spreading and drying on the microscope slide.

At the beginning of the experiments, the spore preparation showed less than 1% of the spores pre-germinated. By 17 h, the control slides had extensive vegetative growth, while no vegetative growth was observed for spores treated with lecithin or linolenic acid. Lecithin and linolenic acid resulted in approximately 67% and 5% inhibition of spore germination, respectively. Thus, lecithin inhibits spore germination and vegetative growth. Linolenic acid partially inhibits germination and inhibits vegetative growth.

The effect of lecithin and linolenic acid on growth also was assessed with Eubacterium rectale. A suspension of Eubacterium rectale (10⁴ cfu/mL) was placed in growth medium and growth was assessed in the presence and absence of lecithin and linolenic acid. Growth of Eubacterium rectale was similar in the control and with cells treated with 1.3 mM lecithin in the growth medium. A few of the cells treated with lecithin were distorted with bulbous enlargement. Growth of Eubacterium rectale was inhibited by 3.3 mM linolenic acid in the growth medium.

### EXAMPLE 3

### Inducing Germination of Spores in the Presence of Antibiotic

Broth (i.e., growth medium as above but without agar) cultures containing C. difficile spores (ca. 10⁴ cfu/mL final concentration) are treated with antibiotics and germinants. Anaerobic conditions at 35° C. on a rotating incubator (10 rpm) are used with a typical experiment including four replications as follows: a) control; b) addition of antibiotic, e.g. cefoxitin, rifaximin, or nitazoxanide, or metronidazole or vancomycin; c) Addition of L-alanine; and d) addition of L-alanine+antibiotic.

Aliquots of 10 μl of broth culture are diluted to be well below the minimum inhibitory concentration (MIC) of the antibiotic, then placed on a microscope slide for immediate (less than 1 h) determination of spore germination or growth. The slide is photographed then and at 8 h intervals, aliquots are sampled to provide quantitation of germination and growth. The sampling duration can be 24 h as the anticipated treatment with the germinant may be daily for four days, with increasing doses of L-alanine daily during the course of antibiotic therapy. An increasing dose protocol can be used to prevent excessively rapid germination that may result in a surge of toxin production.

### EXAMPLE 4

### Broth Culture Testing of Germination Inhibitors

As indicated in Example 2, lecithin and linolenic acid are both inhibitors of spore germination. The testing of Example 2 can be repeated using broth cultures. Broth cultures containing C. difficile spores (ca. 10⁴ cfu/mL final concentration) are treated with germination inhibitors. Anaerobic conditions at 35° C. on a rotating incubator (10 rpm) are used with a typical experiment including four replications as follows: a) Control; b) Addition of lecithin in water; c) addition of linoleic acid sonicated in water; d) addition of linolenic acid sonicated in water; e) addition of linoleic acid with lecithin in water; and f) addition of linolenic acid with lecithin in water. Linoleic acid and linolenic acid do not have sufficient solubility in water to allow dispersion in broth without sonication.

Soy lecithin and egg lecithin can be compared as inhibitors of germination. Soy lecithin contains a higher concentration of linoleic acid (55-60%) and linolenic acid (6-9%) than egg lecithin (15-18% linoleic acid, <1% linolenic). Data are collected using multiple random field photographs (phase microscopy) to allow for quantitation of ungerminated spores, germinated spores, and vegetative cell growth. These data will provide the information necessary to determine the optimal compounds for inhibition of germination and growth and allow subsequent determination of effective concentrations.

Vegetative cell growth is expected in the control. A reduction of growth is expected after treatment with linoleic, linolenic acid, and/or lecithin. Reduction of growth is calculated as a difference in spore germination and/or change in vegetative in cell numbers as compared to the control. A Petroff-Hauser slide is used to determine cfu/mL.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a patient infected with spore-forming bacteria, said method comprising administering to said patient an antibiotic and a spore germinant in amounts and for durations effective for treating said patient.

2. The method of claim 1, wherein said spore-forming bacteria are Clostridium difficile or Clostridium perfringens.

3. The method of claim 1, wherein said spore germinant is an amino acid or derivative thereof.

4. The method of claim 3, wherein said amino acid is selected from the group consisting of L-alanine, L-asparagine, L-cysteine, and L-glutamine

5. The method of claim 3, wherein said spore germinant is N-(L-α-aspartyl)-L-phenylalanine.

6. The method of claim 1, wherein said spore germinant is L-lactate or lactose.

7. The method of claim 1, wherein said spore germinant is carbonic acid.

8. The method of claim 1, wherein said spore germinant is a compound that adsorbs lipids.

9. The method of claim 8, wherein said compound is a starch or charcoal.

10. The method of claim 1, wherein said spore germinant is a bile salt.

11. The method of claim 10, wherein said bile salt is taurocholate.

12. The method of claim 1, wherein said spore germinant is inosine.

13. The method of claim 1, wherein said spore germinant is a mixture of L-alanine, L-lactose, and sodium bicarbonate.

14. The method of claim 1, wherein said antibiotic is vancomycin or metronidazole.

15. The method of claim 1, wherein said antibiotic and said spore germinant are administered simultaneously.

16. The method of claim 1, wherein said antibiotic is administered first and said spore germinant is administered second.

17. The method of claim 1, wherein said spore germinant is administered first and said antibiotic is administered second.

18. The method of claim 1, wherein said spore germinant is administered in increasing amounts over a duration of four days.

19. The method of claim 2, said method further comprising administering to said patient a) an amount of an agent effective for inhibiting Clostridum difficile toxin production or b) an amount of an agent effective for binding and eliminating Clostridum difficile toxin from said patient.

20. The method of claim 19, wherein said agent effective for inhibiting Clostridum difficile toxin production is calcium acetate, calcium gluconate, or calcium saccharate.

21. The method of claim 19, wherein said agent effective for binding and eliminating Clostridum difficile toxin is Tolevamer or Cholestyramine.

22. The method of claim 2, said method further comprising administering a probiotic organism to said patient.

23. The method of claim 22, wherein said probiotic organism is Lactobacillus, Lactococcus, Pediococcus, or Saccharomyces boulardii.

24. The method of claim 18, further comprising administering to said patient an agent effective for inhibiting spore germination, wherein said agent is administered after discontinuing administration of said spore germinant.

25. The method of claim 24, wherein said agent effective for inhibiting spore germination is lecithin, linolenic acid, or sorbate.

26. The method of claim 24, wherein said agent effective for inhibiting spore germination is 1-kestose.

27. A method of preventing Clostridium difficile associated disease (CDAD) in a patient receiving an antibiotic, said method comprising administering a spore germinant to said patient in an amount and for a duration effective to prevent CDAD.

28. The method of claim 27, wherein said spore germinant is L-alanine.

29. The method of claim 27, wherein said method further comprises controlling said patient's diet.

30. The method of claim 27, said method further comprising monitoring said patient for symptoms of CDAD.

31. A method of preventing CDAD in a patient receiving an antibiotic, said method comprising administering an inhibitor of spore germination to said patient in an amount and for a duration effective to prevent CDAD.

32. A composition comprising an antibiotic and a spore germinant.

33. The composition of claim 27, wherein said spore germinant is L-alanine.

34. The composition of claim 32, wherein said antibiotic is vancomycin or metronidazole.

35. The composition of claim 32, said composition further comprising an agent effective for inhibiting Clostridium difficile toxin production or an agent effective for binding and eliminating Clostridium difficile toxin from said patient.

36. An article of manufacture comprising an antibiotic, a spore germinant, and a label or package insert indicating that said antibiotic and spore germinant are useful for preventing CDAD in patient at risk for developing CDAD.

37. The article of manufacture of claim 36, said article of manufacture further comprising an agent effective for inhibiting Clostridium difficile toxin production or an agent effective for binding and eliminating Clostridium difficile toxin from said patient.

38. The article of manufacture of claim 36, said article of manufacture further comprising an agent effective for inhibiting spore germination.

39. A method for treating a patient infected with spore-forming bacteria, said method comprising administering to said patient an antibiotic and L-alanine in amounts and for durations effective for treating said patient.
